(19) United States
(12) Patent Application Publication
(10) Pub. No.: US 2009/0018412 A1
(43) Pub. Date: Jan. 15, 2009

(54) Medical unit with an apparatus for an examination of a patient and an associated method

(75) Inventor: Thomas Schmitt, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/218,070

(22) Filed: Jul. 10, 2008

(30) Foreign Application Priority Data
Jul. 12, 2007 (DE) 10 2007 032 541.1

(51) Int. Cl. A61B 5/00

(52) U.S. Cl. 600/309

(57) ABSTRACT

The invention relates to a medical unit with an apparatus designed for the examination or treatment of a patient. The medical unit comprises at least one means designed for the collection or analysis of at least one substance exuded from the body of the patient during the course of a bodily process concomitantly to the examination or treatment of the patient with the apparatus.

### CROSS REFERENCE TO RELATED APPLICATIONS

This application claims priority of German application No. 10 2007 032 541.1 filed Jul. 12, 2007, which is incorporated by reference herein in its entirety.

### FIELD OF THE INVENTION

The invention relates to a medical unit with an apparatus designed for an examination or treatment of a patient and an associated method.

### BACKGROUND OF THE INVENTION

Medical units generally comprise apparatuses with which it is possible to examine or treat patients. Examples of examination or treatment apparatuses of this kind include X-ray devices, computerized tomographs, magnetic resonance imaging tomographs, ultrasound devices and hybrid modalities for different examination methods and the like. Apparatuses of this kind are used inter alia in the field of disease identification, since it is possible, for example, to use images to draw conclusions, for example, relating to the presence of a cancerous disease and the like.

However, examination and treatment apparatuses of this type only identify diseases in regions of the body of the patient which are, for example, identifiable in an image produced by the examination apparatus or in which there are indications of the presence of diseases. It is possible that further diseases will not be identified.

In addition, the use of examination apparatuses or treatment apparatuses for screening is often associated with the problem that a plurality of data is obtained which the person entrusted with the evaluation, such as a doctor, has only a short time to examine or which, due to the plurality of similar data to be evaluated, is not evaluated with the necessary care. This can result in even the diseases for which the examination was originally intended being overlooked.

### SUMMARY OF THE INVENTION

The invention is therefore based on the object of disclosing a medical unit improved in this regard.

To achieve this object, a medical unit is provided with an apparatus designed for the examination and/or treatment of at least one patient characterized in that the medical unit comprises at least one means designed for the collection and/or analysis of at least one substance exuded from the body of a patient during the course of a bodily process concomitantly to the examination and/or treatment of the patient with the apparatus.

Therefore, the medical unit comprises not only the examination or treatment apparatus, but also one or a plurality of means, with which, in parallel to the examination or treatment with the apparatus, that is simultaneously or shortly beforehand and/or afterward and/or in pauses, the collection and/or analysis of a bodily substance exuded by the patient can be performed. The means is/are also therefore designed to be able to collect and optionally even analyze the substances exuded by the patient during the course of a bodily process during the ongoing examination or treatment with the apparatus.

The fact that the substance is collected should thereby be understood in the broadest sense as meaning that the substance as such may only be collected briefly, for example briefly comes into contact with a region of the means and/or enters into (chemical) reactions with active ingredients of the means or remains permanently in the means (for example in a collection container).

For example, a means can be provided in the form of a sensor which collects odorous substances and analyzes their composition. Due to this additional means, in addition to the apparatus for the examination or treatment, another possibility is available for analyzing measuring data relating to the patient during the course of the identification of a disease or production of a diagnosis. Thereby, during the course of the invention, optionally a wide variety of substances taken from the body of the patient are analyzed. Examples to name include odorous substances exuded by the patient via the respiratory air or the skin or the patient's sweat, hormones, neurotransmitters and other substances emitted on the skin or in bodily orifices and certain regions of the body.

This enables the identification rate to be increased, for example, during the course of a screening or a targeted individual examination.

According to the invention, at least one means of the medical unit can be designed for the collection and/or analysis of at least one odorous substance and/or the respiratory air of the patient and/or of sweat and/or saliva and/or at least one hormone and/or neurotransmitter as a substance exuded from the body of the patient, in particular for the collection and/or analysis of at least one disease-related substance and/or substance that has specifically changed in the presence of a disease.

The means is/are, therefore, means, with which odorous substances or specifically the respiratory air of the patient, sweat and/or an excreted hormone or a plurality of these substances, which originate from the patient's bodily processes, are collected or analyzed. Hereby, it is also possible for the means only to perform an analysis without the direct collection of the exuded substance in the narrower sense being required. For example it is conceivable that it will be sufficient for the respiratory air of the patient only to flow through suitable means.

Therefore, the invention makes use of the fact that, for example, the respiratory air is specifically changed with various diseases. For example, cancer cells form volatile organic compounds which are excreted in the breath in many cases. With suitable sensors, it is then possible, to draw conclusions regarding the presence of skin cancer, bladder cancer or lung cancer or breast cancer. In addition, for example, diabetic patients with low sugar levels form acetone in their respiratory air, while patients with acute kidney failure excrete dimethylamine in the respiratory air. With a suitable means for the identification of odorous substances of this kind or for the analysis of the molecular composition, the respiratory air or other bodily substances can therefore facilitate a direct identification of the disease. The means can be used to send a suitable message for example to a unit for computer-aided diagnosis (CAD) so that a person performing an examination immediately receives, for example on a screen, an indication that, for example, there is a 99% probability that breast cancer or lung cancer is present.

Correspondingly, at least one means can be an odor sensor and/or a respiratory air sensor and/or a fluid sensor and/or a spectroscopic means and/or spectrometric means. For example, it is possible to use silicon sensors comprising components with different coatings with the coatings differing in their chemical or physical properties so that odorous substances are bound (and so collected) with different degrees of effectiveness and correspondingly detection is possible. It is also possible to use chips which work with light-sensitive measuring sensors coated in such a way that their properties can change with respect to the light transmission. It is also possible to use other sensor methods, for example different spectroscopic and spectrometric methods and fluid sensors and the like.

The apparatus designed for the examination and/or treatment of at least one patient can be at least one X-ray apparatus, in particular an X-ray imaging station and/or a fluoroscopic apparatus, and/or a computerized tomography apparatus and/or a magnetic resonance imaging apparatus and/or a positron emission tomography apparatus and/or an ultrasound apparatus and/or a mammography apparatus and/or a dentistry apparatus and/or an endoscopic apparatus and/or a catheter-based apparatus and/or an apparatus from the field of ear, nose and throat medicine. Therefore, the examination or treatment apparatus can be a wide variety of imaging apparatuses and apparatuses with which treatments can be performed. Optionally, a medical unit comprising one or a plurality of means for the analysis of the bodily substances from a patient, can in addition permit the performance of a plurality of different examination or treatment methods, for example with a combined apparatus for the performance of computerized tomography and positron emission tomography or a plurality of X-ray methods. The means for the collection and/or analysis of the bodily substance can, therefore, be combined with a wide variety of treatment and examination apparatuses used in the medical field. Hereby, the means for the collection or analysis of the substance exuded by the patient can be at least partially integrated in the examination or treatment apparatuses or arranged in the vicinity thereof. Optionally, a medical unit according to the invention can comprise an apparatus with a plurality of examination or treatment sites which are each designed with one or a plurality of means for the collection or analysis of the bodily substances.

At least one means can be designed for the automatic, in particular controlled by means of a computational unit (in the unit or the means), collection and/or analysis of at least one substance exuded from the body of the patient during the course of a bodily process. For example, the collection of the respiratory air or the analysis of the respiratory air of a patient can be requested or started with the initiation of an imaging operation of an X-ray unit. This can, for example, take place directly if a measuring protocol for the imaging is started by means of a control unit for the examination and treatment apparatus. On the other hand, automatic collection or analysis of the bodily substance of the patient can take place in that an analysis of this kind is simply performed automatically by the means at specific time intervals or in that the means permanently collects and optionally analyzes any bodily substances exuded for example into a suitable container and/or a specific collection area. In the case of substance collection or substance analysis automatically initiated by means of a computational unit for controlling the examination or treatment apparatus, this does not have to take place directly at the start of the examination or treatment, but, depending on the requirement, can also be performed in the middle of, or in a pause in, the examination or treatment operation. Optionally the collection and/or analysis of the bodily substance can also take place in such a way that the patient's bodily substance is collected over a specific period during the examination and treatment with the apparatus, i.e., for example, the respiratory air from a plurality of breaths, in order to be evaluated subsequently (for example, when a specific quantity of the substance is available).

With particular advantage, at least one result of an analysis of at least one substance exuded from the body of the patient during the course of a bodily process can be transmitted by way of at least one means for the collection and/or analysis at a computational unit for controlling the apparatus for the examination and/or treatment of at least one patient via a data circuit, in particular automatically, and/or determined by means of a computational unit for controlling the apparatus with reference to a collected substance available to the computational unit and/or from a previous analytical result transmitted thereto by the means and optionally displayed on the computational unit on a screen for an operator.

The means, which collects or traps and then analyses the exuded substances, can, therefore, forward the analytical result or the plurality of analytical results obtained to a control unit for example an imaging modality. This can take place over a data circuit, for example over a data line or a radio-based connection and the like. The transmission with particular advantage takes place automatically, or optionally, however, in response to a request, for example from a programming means or an operator on a programming means.

In addition, alternatively or supplementarily, an analytical result can be obtained by a control unit of this kind itself. For this, for example, the collected bodily substance can be forwarded by the means or the plurality of means to the computational unit, for example with the help of an operator, who forwards the correspondingly collected substance to an analysis unit of the computational unit or also automatically over suitable lines for air or fluids and the like.

In addition, a first analytical result of the means for the collection and/or analysis of the bodily substance of the patient can be used in the control unit or the computational unit for a further analysis, for example a result indicating which substances are contained in the respiratory air of the patient, optionally in what amounts, in order during the course of a further evaluation or analysis to determine further results, for example, the possible diseases assigned to the spectrum of the respiratory air. These analytical results can be displayed together with the first already available analytical results or on their own on a screen in order in this way to inform an operator. Optionally, suitable indications of analytical results are issued immediately, for example while the imaging is still being performed with an imaging modality so that optionally the further examination or treatment can be adapted according to the analytical result and consequently suitable information is included in a screening or it is possible to perform a targeted search for a cancerous focus and the like or to take any unusual features into consideration.

According to the invention, an analysis of at least one substance exuded from the body of the patient during the course of a bodily process can be performed jointly with and/or during the course of an evaluation of data and/or results of at least one examination and/or treatment with the apparatus for the examination and/or treatment of the patient, in particular by means of a computational unit for controlling the apparatus and/or during the course of a computer-aided diagnosis. Therefore, it is possible, for example, to analyze or evaluate X-ray data or images during the course of a “computer-aided diagnosis” jointly with exuded aromatic substances.

The apparatus for the examination and/or treatment of at least one patient can be controlled in dependence on at least one result of an analysis of at least one substance exuded from the body of the patient during the course of a bodily process, in particular automatically by means of a computational unit. Therefore, a means for the collection and analysis of the bodily substance can be used to obtain at least one analytical result, with, as described above, it being optionally possible for an analytical result of this kind also to be obtained by an assigned computational unit for controlling the apparatus, for example a control unit for an imaging apparatus. This analytical result is then, automatically or after a request by an operator, used in suitable software of the computational unit, for example in organ programs of a generator to control the apparatus or the plurality of modalities of the apparatus for the examination and treatment of the patient suitably. For example, in the case of a suspected cancer, a suitable collection protocol for the generation of X-ray images can be selected which enables a selected search for the cancerous focus or metastases. It is particularly advantageous for a computational unit, particularly for controlling the apparatus for the examination or treatment of the patient, automatically to perform a suitable control in dependence on the analytical result or the plurality of analytical results. For example, specific imaging protocols requested by the computational unit for cancer identification can be automatically started without an operator having to intervene or can only be confirmed by an operator.

As mentioned above, at least one means for the collection and/or analysis of at least one substance exuded from the body of the patient during the course of a bodily process can be integrated in the apparatus for the examination and/or treatment of at least one patient. For example, a breathing sensor can be arranged on an X-ray grid wall stand. In addition, a breathing sensor can be integrated in the examination tubes of a magnetic resonance imaging tomograph or a computer tomograph. Optionally, it is also possible for a plurality of means of this kind to be distributed, for example, over the length of a tube for the computerized tomography or the area of another apparatus. Integration in the apparatus for the treatment or examination enables a space-saving arrangement which does not require any separate assembly work and in which disturbances from the means for the collection or analysis of the bodily substance can be excluded by a suitable arrangement or taken into account in advance for example in control software.

In addition, however it is obviously also possible that, in the unit, all or individual means for the collection or analysis of the bodily substance are only provided in such a way that they are (loosely) assigned to the corresponding examination or treatment apparatus, for example for provision or insertion in the apparatus or placing on a patient table and the like.

With particular advantage, the medical unit can comprise a plurality of means for the collection and/or analysis of at least one substance exuded from the body of the patient during the course of a bodily process, which are arranged in such a way that during the course of the examination and/or treatment with the apparatus, they may be used to collect and/or analyze substances exuded at different points of the body of the patient.

For example, in addition to a means for the analysis of the respiratory air, it is possible to provide a means for the analysis of the sweat or hormones of the skin. In addition, sensor means can perform odor collection or analysis in a wide variety of body regions for example in the area of wounds or open fractures in order in this way to identify for example metastases on bones. Therefore, it is possible to analyze a wide variety of substances exuded by the patient with respect to their molecular structure, in particular in that a plurality of means are used with which substances can be collected or investigated in different regions of the body. Hereby, the collection can be performed directly outside of the body or in bodily orifices such as the oral cavity or also in open wounds and the like.

According to the invention, at least one means can comprise at least one element for the active collection of at least one substance exuded from the body of the patient during the course of a bodily process, in particular one element for the aspiration and/or for the imaging of respiratory air and/or saliva and/or at least one hormone and/or neurotransmitter from a body region and/or of sweat from the skin. Therefore, it is not absolutely necessary for the bodily substances to be collected passively; it is also possible for selective aspiration to take place, for example of air from the oral cavity or from bodily orifices, which can be natural orifices or artificial orifices, for example, for endoscopic treatments and the like. For example, in this way, substances exuded from the body of the patient, such as odorous substances and further substances can be collected during endoscopic examinations for example in the region of the intestine and the esophagus and during dilations in the coronary region and other coronary examinations.

The analysis can hereby be performed directly with the aid of suitably designed means, which are sufficiently small or compact, for example suitably small chips in odor sensors, inside the body, following which the analytical result is transmitted to the exterior over lines or radio connections and the like. In addition or alternatively, an analysis can be performed externally in that the sampled substances or (first) measurements from the sensor means are transmitted, for example, to a control unit or another computational unit which then performs the detailed actual analysis.

The invention also relates to a method in which concomitantly to the examination and/or treatment of a patient with a suitably designed apparatus of a medical unit with at least one means of medical unit at least one bodily substance exuded from the body of the patient during the course of a bodily process is collected and/or analyzed, in particular with a medical unit as described above. Therefore, with the method advantageously a medical unit is used which, in addition to an apparatus for the examination or treatment of a patient, comprises means for the collection and/or analysis of bodily substances exuded by the patient during the course of at least one bodily process. Therefore, an analysis of the physical or chemical properties of the exuded substances takes place which is performed during the course of an examination or treatment executed as a matter of course, for example the recording of image data or general measurements. Hereby, the subject matter of the method is not the actual examination or treatment, but the simultaneous collection of data relating to an exuded bodily substance such as respiratory air, sweat or the like.

### BRIEF DESCRIPTION OF THE DRAWINGS

Further advantages, features and details of the invention are revealed by the following exemplary embodiments and the drawings, which show:

FIG. 1 a medical unit according to the invention with an X-ray wall device and a breathing sensor,

FIG. 2 a further medical unit according to the invention with an X-ray apparatus and an odor sensor,

FIG. 3 a medical unit with an apparatus for the combined performance of computerized tomography and positron emission tomography with a plurality of means for odor analysis,

FIG. 4 a medical unit with a dentistry apparatus with different devices each with means for the collection of respiratory air and/or saliva,

FIG. 5 a medical unit with a mammography apparatus with a respiratory air sensor and an odor sensor,

FIG. 6 a medical unit with an ear, nose and throat visualization system with an integrated means for the collection and analysis of bodily substances,

FIG. 7 a medical unit with a magnetic resonance imaging apparatus with a plurality of odor and sweat sensors and

FIG. 8 a diagram showing the sequence of a method according to the invention.

### DETAILED DESCRIPTION OF THE INVENTION

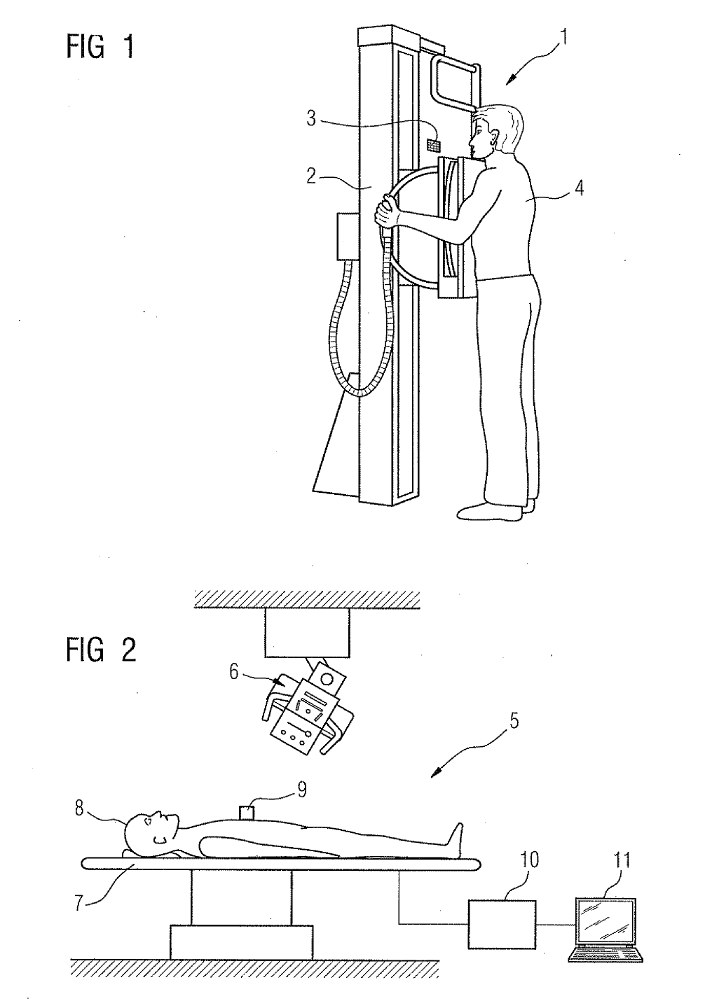

FIG. 1 shows a medical unit according to the invention 1 with an X-ray grid wall device 2 and with a breathing sensor 3 integrated therein. During the performance of the X-ray examination, the patient 4 stands in front of the X-ray grid wall device 2 in such a way that the air exhaled by the patient 4 reaches the region of the breathing sensor 3 and is collected thereby. Optionally, naturally, the patient can also sit down or be supported in any other way. In the breathing sensor 3, a control unit (not shown in further detail here) for the medical unit 1, which also controls the image data recording and evaluation of the measured data from the X-ray grid wall device 2, a molecular analysis of the respiratory air of the patient 4 is performed. This respiratory air analysis may be used to draw conclusions, for example, with respect to the existence of cancerous diseases which are then displayed as indications to an operator on a screen of the control unit for the unit or the X-ray grid wall device 2, who can correspondingly request further images to be taken with the X-ray grid wall device 2 and optionally further examination and treatment apparatuses or selectively evaluate the images of the X-ray grid wall device 2 in a suitable manner.

This enables the identification rate of specific diseases, in particular cancerous diseases, to be significantly increased with the aid of the integrated breathing sensor 3.

FIG. 2 shows a further medical unit according to the invention 5 comprising an X-ray apparatus 6 with a patient bed 7 and with a patient lying thereon 8 and an odor sensor 9. The X-ray apparatus 6 is used to produce images of the patient 8 which are then evaluated automatically by means of a computational unit 10 and/or by an operator. In addition, the odor sensor 9 of the medical unit 5 produces measurements relating to the odor of the skin or, if appropriate, open points of the body (for example in the case of open fractures) of the patient 8, which are also processed by means of the computational unit 10 of the medical unit 1. A screen 11 with an input apparatus for an operator displays analytical results together with the results of imaging by means of the X-ray apparatus 6. In addition, if required, depending upon the analytical results of the measurements made by the odor sensor 9, the computational unit 10 automatically suggests further examination protocols, with which, in the event of a specific disease being suspected, further selective imaging is enabled permitting an as precise as possible determination of the disease of the patient 8 during the later course of the diagnosis.

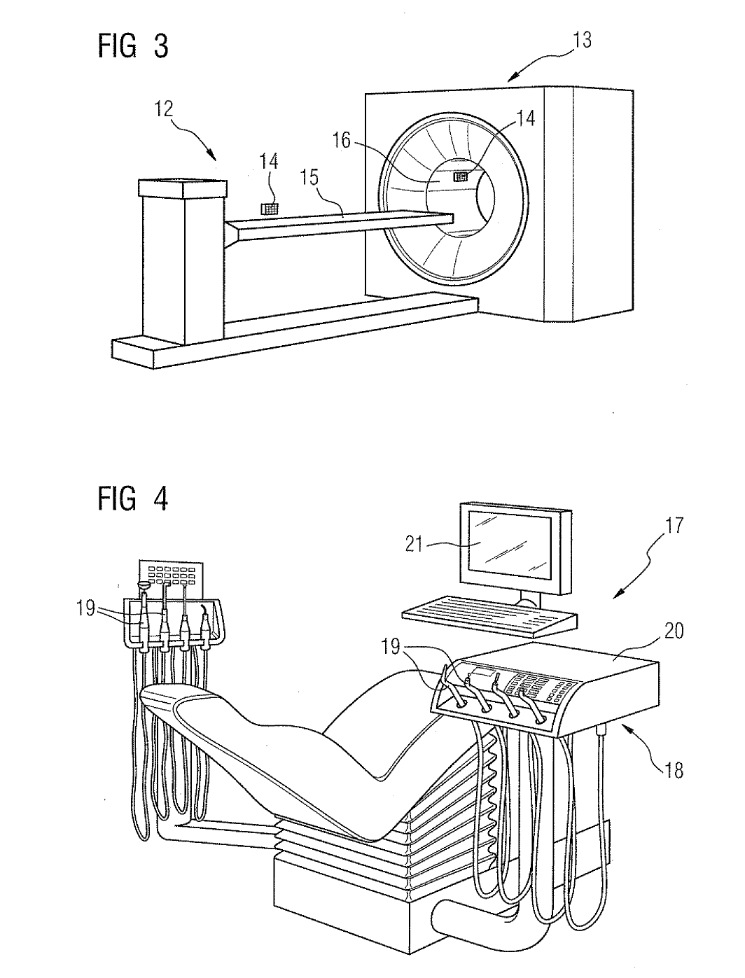

FIG. 3 shows a medical unit 12 comprising an apparatus 13 for the combined performance of computerized tomography and positron emission tomography with a plurality of means 14 for odor analysis. Therefore, the apparatus 13 generates images of a patient (not shown here) on a patient bed 15, whereby, concomitantly to the performance of the examination of the patient on the patient bed 15, with the aid of the means 14 for odor analysis an analysis of the odor molecules in the skin or in the respiratory air of the patient on the patient bed 15 is performed. The means 14 for odor analysis are integrated, on the one hand, in the patient bed 15 and on the other in the tubes 16 of the apparatus 13.

FIG. 4 shows a medical unit 17 with a dentistry apparatus 18 with different devices 19 each with integrated means for the collection of respiratory air and/or saliva (not shown here for reasons of clarity). Samples of the respiratory air or the saliva of a patient (not shown here) are taken by the means for the collection of respiratory air and/or saliva, and depending upon the type of the device 19, forwarded, for example via suitable hoses, to an evaluation unit 20 or directly subjected to a first analysis by the means. The results are displayed together with the results of the dentistry examination or treatment for an operator such as a dentist on a screen 21.

Hereby, the respiratory air analysis or the saliva analysis can also be used to identify a disease which is not directly related to dentistry, for example a lung cancer disease and the like. In such a case, it is possible optionally for a follow-up examination on a further modality of the medical unit 17 or another medical unit to be requested by an operator or automatically initiated.

FIG. 5 shows a medical unit 22 with a mammography apparatus 23 on which are arranged a respiratory air sensor 24 and an odor sensor 25. This enables, simultaneously to the generation of the mammography images with the mammography apparatus 23, a respiratory air analysis of respiratory air of the patient and an odor analysis relating to the odorous substances excreted by the patient via the skin. The existence of an analytical result or the result itself can be displayed directly for example on suitable display fields of the respiratory air sensor 24 or the odor sensor 25 or following transmission to a control unit (not shown here) for the mammography apparatus 23. If the analytical result of the respiratory air sensor 24 or the odor sensor 25 gives rise to suspicion of a cancerous disease, optionally further examinations can be performed automatically or at the request of an operator.

Optionally, for this, the apparatus of the medical unit 22 can comprise further modalities for examination or treatment with which further data images, such as for example ultrasound images or magnetic resonance images are possible.

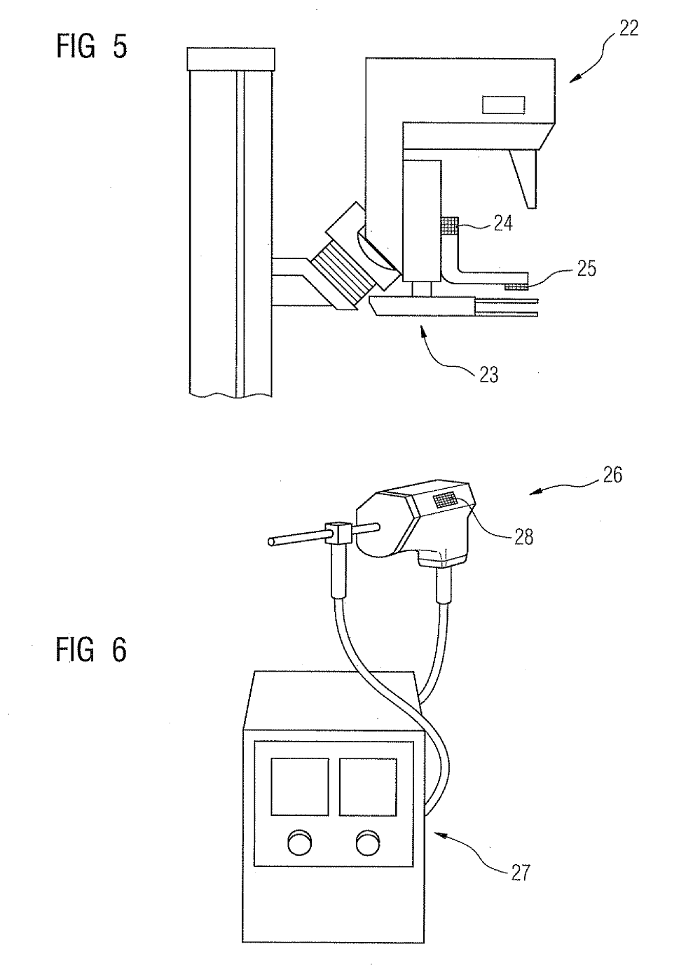

FIG. 6 shows a medical unit 26 with an ear, nose and throat visualization system 27 comprising an integrated respiratory air and odor sensor 28. The sensor is indicated here by a box. This can collect and analyze the respiratory air and odorous substances exuded by the patient during examinations and treatments in the ear, nose and throat regions in order in this way to be able to identify diseases of the patient with a higher detection rate.

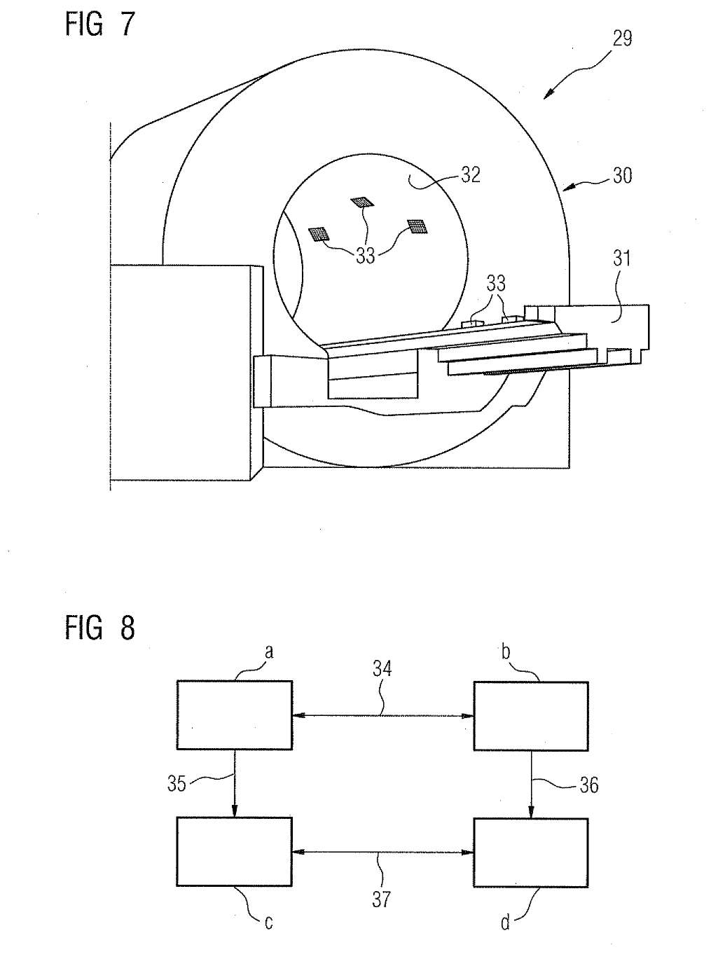

The medical unit 29 in FIG. 7 comprises a magnetic resonance imaging apparatus 30 with a patient bed 31 and a magnetic resonance imaging tube 32, with a plurality of odor and/or sweat sensors 33 being distributed in the region of the patient bed 31 and the magnetic resonance imaging tubes 32, which make it possible to analyze the odorous substances, excreted via the skin or open wounds and bodily orifices of the patient, and the sweat of the patient with regard to their molecular composition. This takes place during the course of computer-aided diagnosis with the aid of a control unit of the medical unit 29 (not shown here), via which the protocol selection for the magnetic resonance images is also performed, which is adapted optionally automatically or confirmed by an operator in dependence on a first analytical result of the odor and sweat sensor 33.

This means that subsequently, optionally also automatically, it is possible for a successful diagnosis to be performed much more reliably and possibly without the need for further separate examinations.

FIG. 8 finally shows a diagram of the sequence of a method according to the invention in which the box a represents the examination and/or treatment of a patient with an apparatus such as a magnetic resonance imaging tomograph, an X-ray apparatus and the like. The box b signifies the collection and/or analysis of a substance exuded from the body of the patient or plurality of such substances during the course of a bodily process by suitable means.

These two procedures take place in parallel, that is substantially simultaneously or at least in one operation, as indicated by the double-headed arrow 34. The box c symbolizes the examination and/or treatment results during the course of the examination and/or treatment according to the arrow 35, while the box d stands for the analytical results obtained from the collection and/or analysis of the bodily substances according to box b according to arrow 36.

The examination and/or treatment results according to box c and the analytical results according to box d can be related to each other, for example to the effect that images from examination and/or treatment according to box a are selectively analyzed with regard to a suspected cancer determined from the analytical result according to box d for example by a programming means. This interrelationship or the establishment of suitable connections are indicated by the arrow 37. This permits a substantially better identification of unusual features or features during the course of the method according to the invention or diseases. Therefore, the method provides selective support for an automatic diagnosis or a later diagnosis made by means of a doctor by a suitable analysis of measurements, which can be performed for example automatically by a suitable control unit of a medical unit used with a method according to the invention of this kind.

1.-12. (canceled)

13. A medical unit, comprising:
an apparatus that examines a patient; and
a unit that collects a substance exuded from a body of the patient during a course of a bodily process of the patient concomitantly to the examination by the apparatus.

14. The medical unit as claimed in claim 13, wherein the substance comprises a disease-related substance or a substance specifically changed in a disease.

15. The medical unit as claimed in claim 13, wherein the substance is selected from the group consisting of: odorous, respiratory air, sweat, saliva, hormone, and neurotransmitter.

16. The medical unit as claimed in claim 13, wherein the unit is selected from the group consisting of: an odor sensor, a respiratory air sensor, a fluid sensor, a spectroscopic unit, and a spectrometric unit.

17. The medical unit as claimed in claim 13, wherein the unit analyzes the collected substance during the course of the bodily process of the patient concomitantly to the examination by the apparatus.

18. The medical unit as claimed in claim 13, wherein the unit is controlled by a computational unit for collecting or analyzing the substance.

19. The medical unit as claimed in claim 18, wherein the unit is controlled by the computational unit for automatically collecting or analyzing the substance.

20. The medical unit as claimed in claim 13, wherein a result of an analysis of the substance is transmitted to a computational unit.

21. The medical unit as claimed in claim 13, wherein the apparatus is controlled by a computational unit based on a result of an analysis of the substance.

22. The medical unit as claimed in claim 21, wherein the apparatus is automatically controlled by the computational unit based on the result of the analysis of the substance.

23. The medical unit as claimed in claim 21, wherein the result is optionally displayed on a screen on the computational unit for an operator.

24. The medical unit as claimed in claim 13, wherein the substance is jointly analyzed during an evaluation of an examination data or a treatment data of the patient by a computational unit.

25. The medical unit as claimed in claim 13, wherein the unit is integrated in the apparatus.

26. The medical unit as claimed in claim 13, further comprising a plurality of units for collecting or analyzing the substance exuded from a plurality of different points of the body of the patient.

27. The medical unit as claimed in claim 13, wherein the apparatus performs a treatment for the patient.

28. The medical unit as claimed in claim 13, wherein the apparatus is selected from the group consisting of: an X-ray apparatus, a computerized tomography apparatus, a magnetic resonance imaging apparatus, a positron emission tomography apparatus, an ultrasound apparatus, a mammography apparatus, a dentistry apparatus, an endoscopic apparatus, a catheter-based apparatus, an ear medicine apparatus, a nose medicine apparatus, and a throat medicine apparatus.

29. A method for assisting an examination of a patient, comprising:
collecting a substance exuded from a body of the patient during a course of a bodily process of the patient concomitantly to the examination; and
examining the patient based on the collected substance.
